(19) United States
(12) Patent Application Publication
(10) Pub. No.: US 2003/0153771 A1
(43) Pub. Date: Aug. 14, 2003

(54) Large scale synthesis of optically pure aziridines

(76) Inventors: Hartmuth C. Kolb, San Diego, CA (US); Ramanaiah C. Kanamarlapudi, Bridgewater, NJ (US); Paul F. Richardson, Monmouth Junction, NJ (US); Gaznabi Khan, Caldwell, NJ (US)

Correspondence Address: McDonnell Boehnen Hulbert & Berghoff, 32nd Floor, 300 S. Wacker Drive, Chicago, IL, 60606, US

(21) Appl. No.: 10/256,599

(22) Filed: Sep. 27, 2002

Related U.S. Application Data
(60) Provisional application No. 60/325,469, filed on Sep. 27, 2001.

(51) Int. Cl. C07D 203/04

(52) U.S. Cl. 548/954

(57) ABSTRACT

Disclosed is an efficient, inexpensive method for the preparation of chiral aziridines on a large scale.

### BACKGROUND OF THE INVENTION

1. Field of the Invention

This invention relates to a practical method for preparing optically pure aziridines from 1,2-aminoalcohols.

2. Description of the Related Art

Chiral aziridines represent a class of highly desirable synthetic intermediates (See Mc Coull, W. et al., Synthesis 2000, 1347; Tanner, D. Angew. Chem. Int. Ed. Engl. 1994, 33, 599; and Tanner, D. Pure and Appl. Chem. 1993, 1319). As such, a great amount of work has been expounded in devising methods for their synthesis (Osborn, H. M. I. et al., Tet. Asymm. 1997, 8, 1693).

Several groups have concentrated on developing metal catalysed aziridination systems, which transfer an imido group to an olefin to yield the optically active aziridine protected as a sulfonamide. This methodology has undergone significant improvements over the past several years both in terms of process and enantioselectivity. The most commonly used systems are based on copper, manganese and rhodium, but a number of permutations of catalyst, ligand, and substrate scope exist (See Minakata, S. et al., Angew Chem. Int. Ed. Engl., 1998, 37, 3392; Nishikori, H. et al., Tet. Lett., 1996, 37, 9245; Li, Z. et al., J. Am. Chem. Soc., 1993, 115, 5326; Evans, D. A. et al., J. Am. Chem. Soc., 1993, 115, 5328; and Li, Z. et al., J. Am. Chem. Soc., 1995, 117, 5889). However, several significant drawbacks still exist, which limit this method as a useful synthetic tool. For instance, the reaction is highly substrate specific. Secondly, the enantioselectivity is dependent on the nature of the imido source and thus the reaction is limited to which sulfonamide group can be placed on the aziridine. Finally, the major problem in this area is finding a useful nitrene source (Sodergren, M. J. et al., Tet.Asym. 1997, 8, 3563; Sodergren, M. J. et al., Tet.Lett. 1997, 38, 6897). The most commonly used source to date has been N-p-(tosylsulfonyl)iminophenyliodinate. However, this reagent is very expensive, very insoluble and generates a relatively heavy stoichiometric by-product (iodobenzene).

Various other approaches have been adopted for the production of chiral aziridines (Mori, K. et al., Tet.Asym. 1990, 1, 281.; Martres, M. et al., Tet. Lett., 1994, 35, 8787). In general these all involve synthetic manipulation of readily available chiral intermediates. By far, the most commonly employed reaction is the formal dehydration of chiral amino alcohols, which are obtained by reducing the commercially available amino acid to yield the aziridine. Several reagents have been developed for this method (Kelly, J. W. et al., J. Org.Chem. 1986, 51, 95), and a Mitsunobu-type reaction can also be employed (Wipf, P. et al., Tet.Lett., 1992, 33, 6267; Pfister, J. R., Synthesis, 1994, 969). However, these methods are not often amenable to scale up.

Another method reports a three-step method to prepare N-tosylsulfonamide protected aziridines starting from the amino acid (Craig D., Synlett. 1992, 4). Protection of the amino functionality is carried out prior to reduction and cyclization, thereby circumventing the difficulties encountered with isolation of the amino-alcohol from the reduction of the amino acid. However, this methodology can not be used to form a wide range of different sulfonamides. Also, deprotection of sulfonyl aziridines often requires harsh conditions and results in low yields (Alonso, D. et al., J. Org. Chem. 1998, 63, 9455). Even the mild Fukuyama conditions (Tet. Lett. 1995, 36, 6373 and Tet. Lett. 1997, 33, 5831) for removal of a nosyl group invariably fail, leading to ring opening products.

Reduction of amino acid compounds to the chiral amino alcohols can be carried out on a large scale using the safe and economical conditions reported by Masamune (Abiko, A. et al., Tet.Lett. 1992, 33, 5517). The classical method to cyclize an amino-alcohol is first reported by Wenker in 1935 (Wenker, H., J. Am.Chem.Soc., 1935, 57, 2328). A similar methodology has been used for the synthesis of optically active polymers (Minoura, Y. et al., J.Am.Chem.Soc., 1959, 81, 4689). Also, Kostyanovsky uses a similar procedure for the syntheses of N-halo-2-alkylaziridines (Kostyanovsky, R. G. et al., Tet. Lett. 1969, 9, 719). In neither either case is the Wenker cyclisation of a series of amino alcohols disclosed or suggested or the enantiomeric excess rigorously determined.

It is therefore an object of the invention to provide a method for the preparation of chiral aziridines that is easily amenable to scale-up.

It is another object of the invention to provide a method for the preparation of chiral aziridines that employs cheap starting materials and reagents.

It is further an object of the invention to provide an efficient method for the preparation of chiral unprotected aziridines.

It is further an object of the invention to provide an efficient method for the preparation of chiral protected aziridines.

### SUMMARY OF THE INVENTION

The present invention provides for a method of preparing chiral aziridines from corresponding α-amino alcohols. Specifically, an α-amino alcohol is treated with sulfuric acid to form a sulfate ester and the sulfate ester is subsequently cyclized to the chiral aziridine in the presence of base. Chiral aziridines are known in the art as useful synthetic precursors (Mc Coull, W. et al., Synthesis 2000, 1347; Tanner, D. Angew. Chem. Int. Ed. Engl. 1994, 33, 599; and Tanner, D. Pure and Appl. Chem. 1993, 1319, all incorporated herein by reference).

The present invention also provides for a method of converting these chiral aziridines into sulfonamides by treatment with the corresponding sulfonyl chloride. This simple process gives N-sulfonyl protected optically pure aziridines in high yield and purity after a simple work-up.

### DETAILED DESCRIPTION OF THE INVENTION

According to Scheme 1, a preferred embodiment of the present invention relates to a method for the formation of a chiral aziridine IV by treating an α-amino alcohol I with sulfuric acid at elevated temperature and reduced pressure and then cyclizing the resulting sulfate ester III at elevated temperature in the presence of base. The conversion of the amino alcohol I to the sulfate ester III proceeds through the sulfate II.

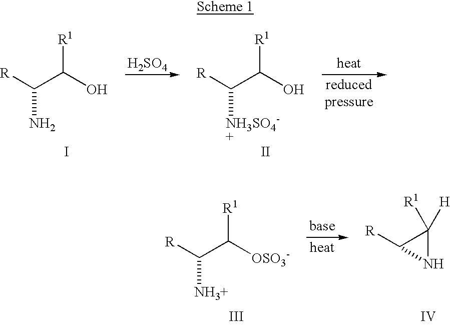

In Scheme 1:

[symbol] is a bond representing either (S) or (R) configuration with respect to the carbon atom to which it is attached;

R is hydrogen, or

lower alkyl optionally substituted with one, two, three or four groups independently selected from halogen, aryl, cycloalkyl, heteroaryl, lower alkoxy, —C(O)-alkyl, —C(O)NH-alkyl, C(O)N-dialkyl, nitro, alkenyl or alkynyl, or

aryl or heteroaryl optionally substituted with one, two, three or four groups independently selected from halogen, lower alkyl, lower alkoxy, —C(O)— alkyl, —C(O)NH-alkyl, C(O)N-dialkyl, nitro, alkenyl or alkynyl;

R¹ is hydrogen or lower alkyl.

In a more preferred embodiment, R is optionally substituted lower alkyl and R¹ is hydrogen.

By “alkyl”, “lower alkyl”, and “Cl-Clo alkyl” in the present invention is meant straight or branched chain alkyl groups having 1-10 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. These groups may be substituted with up to four groups mentioned below for substituted aryl.

By “alkoxy”, “lower alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy. These groups may be substituted with up to four groups mentioned below for substituted aryl.

By the term “halogen” in the present invention is meant fluorine, bromine, chlorine, and iodine.

A “carbocyclic group” or “cycloalkyl” is a nonaromatic cyclic ring or fused rings having from 4 to 7 ring members. Examples include cyclobutyl and cycloheptyl. These rings may be substituted with one or more of the substituent groups mentioned below for aryl, for example alkyl, and alkoxy. Typical substituted carbocyclic groups include 2-chlorocyclopropyl, 2,3-diethoxycyclopentyl, and 2,2,4,4-tetrafluorocyclohexyl. The carbocyclic group may contain one or two heteroatoms selected from oxygen, sulfur, and nitrogen, and such ring systems may be referred to as “heterocyclyl” or “heterocyclic”. These heterocyclyl groups may be substituted with up to four of the substituent groups mentioned for aryl.

By heteroaryl is meant one or more aromatic ring systems of 5-, 6-, or 7-membered rings containing at least one and up to four heteroatoms selected from nitrogen, oxygen, or sulfur. Such heteroaryl groups include, for example, imidazolyl, (is)oxazolyl, pyridyl, pyrimidinyl, (iso)quinolinyl, napthyridinyl, benzimidazolyl, benzoxazolyl. The heteroaryl group is optionally substituted with up to four groups mentioned below for substituted aryl.

By aryl is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., —OH, lower alkyl, lower alkoxy, aryl, heteroaryl, and nitro. A preferred aryl is phenyl.

Additionally, as seen above in Scheme I, the compounds prepared by the instant invention may have one or more asymmetrical carbon atoms and, therefore, may exist in stereoisomeric forms. All stereoisomers are intended to be included within the scope of the present invention. As used, “stereoisomer” or “stereoisomeric” refers to a compound which has the same molecular weight, chemical composition, and constitution as another, but with the atoms grouped such that their orientation in three-dimensional space is different. The method of the present invention focuses on forming compounds in enantiomerically pure form.

In another more preferred embodiment, the amino alcohol I is treated with sulfuric acid with one or more suitable solvents. The reaction mixture in such a solvent can be homogenous or heterogenous. Examples of suitable solvents for the present method include, but are not limited to, one or more of the following: a protic solvent such as methanol, ethanol or water; an aprotic solvents such as dimethylsulfoxide, dimethylformamide or hexamethylphosphorotriamide; or other organic solvents such as, for example, diethyl ether or tetrahydrofuran (THF). In an even more preferred embodiment, the solvent is selected from water or ether.

In still another more preferred embodiment, I is initially reacted with sulfuric acid at temperatures of from between −10° C. and 25° C., preferably at about 5° C. To facilitate the conversion of the sulfate II to the sulfate ester III, the solvent is removed and the reaction is then carried out at temperatures of from between 50° C. and 250° C. Even more preferably, the reaction temperature is from between 100° C. and 200° C. and still even more preferably the reaction temperature is from between 125° C. and 150° C. A most preferred temperature is around 135° C.

The sulfuric acid in the present invention is obtained from commercial sources. Preferably, the sulfuric acid is 95-98% sulfuric acid and even more preferably, the 95-98% sulfuric acid is diluted in solvent, such as, for example, water, which is subsequently removed after the formation of the sulfate II.

Acceptable reaction pressures for the formation of II range from between 0.5 mm/Hg to 10 mm/Hg. In a more preferred embodiment, the reaction pressure is from between 1 and 5 mm/Hg and most preferably around 2 mm/Hg or lower.

In yet another more preferred embodiment, the sulfate ester intermediate III is treated with a base in a suitable solvent. The reaction mixture in such a solvent can be homogenous or heterogenous. In an even more preferred embodiment, the solvent is water, which also aids in the purification of the product IV.

Suitable bases used in the cyclization of III to the chiral aziridine IV are those with alkali metals or alkaline earth metals such as sodium, potassium, calcium and magnesium, and those with organic bases including, but not limited to, amines. Preferred bases are alkali metal bases or alkaline earth metal bases. Even more preferred bases are alkaline metal hydroxides, such as, for example, potassium hydroxide or sodium hydroxide.

As depicted in Scheme 2, in yet another more preferred embodiment, the chiral aziridine IV prepared by the present method is treated with a suitable sulfonyl chloride to produce a chiral N-sulfonylaziridine V.

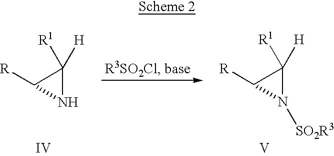

In Scheme 2, R, R¹ and R² are as defined above for Scheme 1, and:

R³ is lower alkyl optionally substituted with one, two or three groups independently selected from halogen, aryl, cycloalkyl, heteroaryl, lower alkoxy, —C(O)-alkyl, —C(O)NH-alkyl, C(O)N-dialkyl, —C(O)O-alkyl, cyano, nitro, alkenyl or alkynyl, or

aryl optionally substituted with one, two, three or four groups independently selected from halogen, lower alkyl, lower alkoxy, —C(O)-alkyl, —C(O)NH— alkyl, C(O)N-dialkyl, —C(O)O-alkyl, cyano, nitro, alkenyl or alkynyl.

The disclosures in this application of all articles and references, including patents, are incorporated herein by reference.

The invention is illustrated further by the following examples which are not to be construed as limiting the invention in scope or spirit to the specific procedures described in them.

The starting materials and various intermediates may be obtained from commercial sources, prepared from commercially available organic compounds, or prepared using well known synthetic methods. Amino alcohols used in the present invention are prepared or purchased from Omega Chemicals.

Representative examples of the invention are set forth below.

### EXAMPLE 1

Synthesis of Amino Alcohols

1. 2-Amino-3-methyl-butan-1-ol

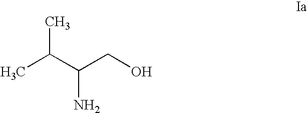

To a stirred suspension of NaBH₄ (1.136 g; 29.9 moles) in THF (12 L) is added DL-valine (1.4 Kg; 11.95 moles). The flask is immersed in an ice-water bath, and a solution of concentrated H₂SO₄ (1,325 g; 14.94 moles; 1.25 eq.) in diethyl ether (total volume of 2.4 L) is added dropwise at such a rate as to maintain the reaction mixture below 20° C. (addition time ˜10 hours). Stirring of the reaction mixture is continued at room temperature overnight, and methanol (1.5 L) is added carefully to destroy excess BH₃. The mixture is stirred for 3 hours at room temperature and then concentrated. 5 L NaOH (8 L; 5 M sol.) is added and the mixture is distilled. The solvent that distills below 100° C. is removed. The mixture is then heated at reflux for 3 hours. The turbid aqueous mixture is cooled and extracted with CH₂Cl₂ (2×5 L) and dried over MgSO₄. Evaporation of the solvent gives crude product which is purified via vacuum distillation (90-95° C./10-15 mmHg) yielding 724 g (59%) of pure 2-Amino-3-methyl-butan-1-ol (a).

2. (S)-2-Amino-3-cyclohexyl-propan-1-ol

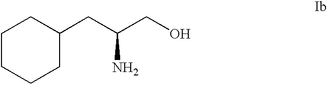

(S)-2-Amino-3-phenyl-propan-1-ol is taken up in a mixture of acetic acid and water (3:2) and subjected to hydrogenation under pressure (60 psi) at 60° C. using platinum oxide as the catalyst. Reduction of the aromatic ring is monitored by ¹H NMR spectroscopy. Upon completion, the unsaturated amino alcohol is purified by vacuum distillation (120° C./1 mm/Hg) to yield the title compound (Ib) as a thick syrup.

### EXAMPLE 2

Synthesis of Chiral Aziridines

1. (S)-2-Benzylaziridine

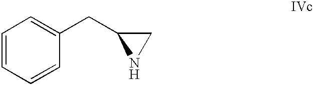

A solution of sulfuric acid (181.8 ml, 95% purity; 3.41 mol) in water (260 ml) is added slowly to a stirred, cooled (5° C.) solution of L-phenylalaninol (500 g, 3.31 mol) in water (800 ml). After the addition is completed, the reaction mixture is stirred for a another hour. The water is removed in vacuo. The residue is then heated to 140° C. under vacuum (2 mm/Hg) for 2 days (the transformation of the hydroxy compound to the sulfate ester can be easily followed by ¹H NMR spectroscopy). The solid is then allowed to cool to room temperature and 30% sodium hydroxide solution (1700 ml) is added. A reflux condenser is fitted to the flask and the solution is heated to 135 C for 6 hours. The mixture is allowed to cool before being extracted with diethyl ether (3×500 ml). The combined organic layers are dried over sodium hydroxide pellets (ca. 100 g) and filtered. The solvent is removed under reduced pressure and the product is purified by vacuum distillation off sodium hydroxide pellets (80-85° C./0.5 mm/Hg) to give 306 g (70.5%) of pure (S)-2-benzylaziridine (IVc) as a colorless liquid. Sodium hydroxide pellets are added as a preservative and the material is stored in the refrigerator.

¹H NMR (CDCl₃): 7.35-7.2 (m, 5H), 2.83, 2.77 (dd, J=6.07 Hz, 6.08 Hz, 1H), 2.68, 2.63 (dd, J=5.95 Hz, 5.93 Hz, 1H), 2.23-2.16 (m, 1H), 1.80 (d, J=5.76 Hz, 1H), 1.44 (d, J=3.49 Hz, 1H). ¹³C NMR (CDCl₃): 139.6, 129.1, 128.7, 126.6, 40.4, 31.2, 25.1.

2. (S)-2-Isopropylaziridine

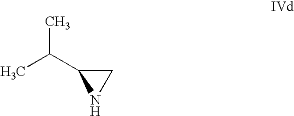

A solution of sulfuric acid (246 ml, 95% purity; 4.618 mol) in water (460 ml) is added slowly to a stirred, cooled (5° C.) solution of L-valinol (500 g, 4.854 mol) in water (540 ml). After the addition is completed, the reaction mixture is stirred for another hour, after which the water is removed in vacuo. The residue is heated to 135° C. under vacuum (2 mm/Hg) for 2 days. During this period, the oil turns slowly into a solid mass (the conversion of the hydroxy compound to the sulfate ester can easily be followed by ¹H NMR). The solid is then allowed to cool to room temperature and 30% sodium hydroxide solution (2000 ml) is added. A reflux condenser is fitted to the flask and the solution is heated to 110° C. for 3 hours. The reflux condenser is replaced with a short path distillation head, and the mixture is subjected to steam distillation. Approximately 1000 ml of distillate is collected in the receiver flask. The distillate is extracted with diethyl ether (3×500 ml). The combined organic layers are dried over sodium hydroxide pellets (100 g) and filtered. The solvent is removed under reduced pressure, and the product is purified by distillation off sodium hydroxide pellets (b.p 104-106° C.) to give 280.1 g (68%) of pure (S)-2-Isopropylaziridine (IVd) as a colorless liquid. The pure aziridine is stored over sodium hydroxide pellets (as a preservative) in the refrigerator.

¹H NMR (CDCl₃): 1.68(m, 2H), 11.31(m, 1H), 1.12 (m, 1H), 1.0 (d, J=5.87 Hz, 3H), 0.93 (d, J=6.24 Hz, 3H). ¹³C NMR(CDCl₃): 37.0, 33.0, 24.1, 20.5, 19.9.

3. (2S)-2-((1S)1-methylpropyl)aziridine

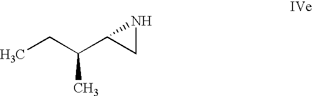

A solution of sulfuric acid (227 ml, 95% purity; 4.264 mol) in water (150 ml) is added slowly to a stirred, cooled (5° C.) solution of L-isoleucinol (500 g, 4.273 mol) in water (480 ml). After the addition is completed, the reaction mixture is stirred for another hour, after which the water is removed in vacuo. The residue is heated to 135° C. under vacuum (2 mm/Hg) for 2 days. During this period, the oil turns slowly into a solid mass (the conversion of the hydroxy compound to the sulfate ester can easily be followed by ¹H NMR). The solid is then allowed to cool to room temperature and 30% sodium hydroxide solution (2000 ml) is added. A reflux condenser is fitted to the flask and the solution is heated to 135° C. for 3 hours. The reflux condenser is replaced with a short path distillation head, and the mixture is subjected to steam distillation. Approximately 1200 ml of distillate is collected in the receiver flask. The distillate is extracted with diethyl ether (3×500 ml). The combined organic layers are dried over sodium hydroxide pellets (100 g) and filtered. The solvent is removed under reduced pressure, and the product is purified by distillation off sodium hydroxide pellets (b.p 125-128° C.) to give 338.8 g (80.1%) of pure (2S)-2-((1S)-l-methylpropyl)aziridine (IVe) as a colorless liquid. The pure aziridine is stored over sodium hydroxide pellets (as a preservative) in the refrigerator.

¹H NMR (CDCl₃): 1.65 (m, 3H), 1.31 (m, 2H), 0.95 (m, 7H). ¹³C NMR (CDCl₃): 39.8, 36.0, 28.5, 23.6, 16.9, 11.4.

4. (S)-2-Cyclohexylaziridine

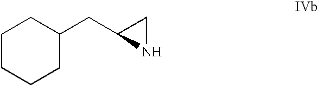

A solution of sulfuric acid (33.9 ml, 95% purity; 0.636 mol) in water (35 ml) is added slowly to a stirred, cooled (5° C.) solution of (S)-2-amino-3-cyclohexyl-propan-1-ol (Ib) (100 g, 0.636 mol) in water (80 ml). After the addition is completed, the reaction mixture is stirred for a further hour, after which the water is removed in vacuo. The residue is heated to 135° C. under vacuum (2 mm/Hg) for 2 days. During this period, the oil turns slowly into a solid mass (the conversion of the hydroxy compound to the sulfate ester can easily be followed by ¹H NMR). The solid is then allowed to cool to room temperature and 30% sodium hydroxide solution (400 ml) is added. A reflux condenser is fitted to the flask and the solution is heated to 135° C. for 3 hours. The reflux condenser is replaced with a short path distillation head, and the mixture is subjected to steam distillation. Approximately 250 ml of distillate is collected in the receiver flask. The distillate is extracted with diethyl ether (3×100 ml). The combined organic layers are dried over sodium hydroxide pellets (10 g) and filtered. The solvent is removed under reduced pressure, and the product is purified by distillation off sodium hydroxide pellets (b.p 60-63° C./1 mm/Hg) to give 54.4 g (62%) of pure (S)-2-cyclohexylaziridine (IVb) as a colorless liquid. The pure aziridine is stored over sodium hydroxide pellets in the refrigerator.

¹H NMR (CDCl₃): 1.92 (m, 1H), 1.65 (m, 6H), 1.42(m, 1H), 1.15(m, 6H), 0.94(m, 2H).

¹³C NMR (CDCl₃): 42.8, 37.3, 34.0, 33.5, 28.9, 26.8, 26.7, 26.6, 25.6.

5. (S)-2-Isobutylaziridine

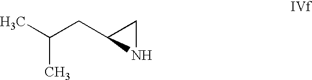

To a chilled (5° C.), stirred solution of (S)-(+)-leucinol (500 g, 4.27 mol) in water (480 ml) is added a solution of concentrated H₂SO₄ (418.5 g, 4.27 mol) in water (150 ml). After the addition is complete, the majority of the water is removed in vacuo. The residual viscous oil is heated to 140° C. under high vacuum (1-2 mm/Hg) for 3 days. During this period the oil slowly turns into a solid mass (sulfate ester). The product is allowed to cool to room temperature and then is pulverized in a mortar. The powder is transferred to a flask and sodium hydroxide solution (902 ml, 30% aqueous) is added. A reflux condenser is fitted to the flask and the solution is refluxed for 3 hours. The reflux condenser is replaced with a short path distillation head, and the mixture is subjected to steam distillation. Approximately 1000 ml of a biphasic distillate is collected in the receiver flask. The upper organic layer is separated and the aqueous phase extracted with ether (2×400 ml). The combined organic phases are dried over sodium hydroxide pellets (125 g) and the solvent removed in vacuo to afford a yellow oil. The product is distilled off sodium hydroxide pellets (20 g) (bp 130° C.) to give 244.9 g (58% yield) of pure (S)-isobutylaziridine (IVf) as a colorless oil. The pure aziridine is stored over sodium hydroxide pellets (as a preservative) in the refrigerator. ¹H NMR (CDCl₃): 1.98-1.88 (m, 1H), 1.86-1.71 (m, 2H), 1.38-1.26(m, 2H), 0.96 (d, J=6.7 Hz, 6H); ¹³C NMR (CDCl₃): 43.8, 28.8, 27.5, 25.1, 23.0, 22.5.

### EXAMPLE 3

Synthesis of Chiral N-Su/fonylaziridines

1. General Procedure

A solution of the (S)-aziridine (0.252 mol) and triethylamine (0.29 mol) in methylene chloride (200 ml) is added dropwise to a solution of the sulfonyl chloride (0.21 mol) in methylene chloride (400 ml) at −30° C. (dry ice/CH₃CN bath) over a period of 60 minutes. The stirring is continued for a further 30 minutes (the consumption of the sulfonyl chloride can be monitored by thin layer chromatography). The cooling bath is removed and the reaction is allowed to warm to 10° C. before being poured into ice water (600 ml). The organic layer is separated and sequentially washed with cold 5% hydrochloric acid (3×100), water (100 ml), saturated sodium bicarbonate solution (3×100 ml), water (100 ml) and brine (100 ml). The organic layer is dried over sodium sulfate, and the solvent removed in vacuo at a temperature of less than 30° C. If a solid appears, concentration is stopped and hexane/diethyl ether (1:1) is slowly added to precipitate the sulfonamide product. In the other cases, the sulfonamide is obtained as a viscous oil after complete evaporation of the volatiles.

The following compounds of are prepared in this matter. The yield ranges from 70-80%.

2. (S)-2-Benzyl-1-(toluene-4-sulfonyl)aziridine

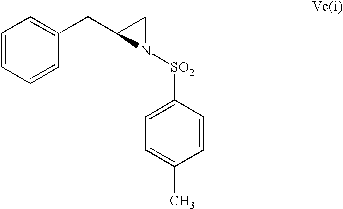

White solid; ¹H NMR (CDCl₃): 7.70 (m, 2H), 7.21 (m, 5H), 7.06 (m, 2H), 2.84 (m, 4H), 2.44 (s, 3H), 2.18(d, J=4.5 Hz, 1H); ¹³C NMR (CDCl₃): 144.7, 137.4, 135.2, 130.0, 129.1, 128.8, 128.2, 126.8, 41.6, 37.8, 33.2, 22.0.

3. (S)-1-Benzenesulfonyl-2-benzyl-aziridine

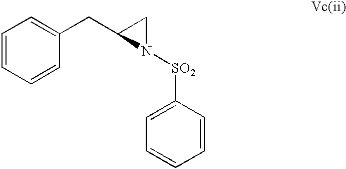

White solid; ¹H NMR (CDCl₃): 7.85 (m, 2H), 7.59(m, 1H), 7.46(m, 2H), 7.18(m, 3H), 7.06(m, 2H), 3.02(m, 1H), 2.83(m, 3H), 2.19(d, J=4.53 Hz, 1H); ¹³C NMR (CDCl₃): 138.3, 137.3, 133.9, 129.4, 129.1, 128.9, 128.2, 127.1, 41.8, 37.8, 33.2.

4. (S)-2-Benzyl-1-(4-methoxybenzenesulfonyl)aziridine

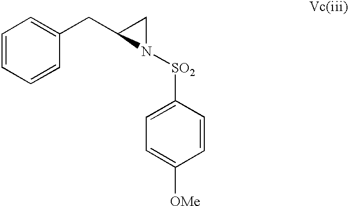

White solid; ¹H NMR (CDCl₃): 7.73 (d, J=8.97 Hz, 2H), 7.16 (m, 3H), 7.06(m, 2H), 6.88(d, J=8.97 Hz, 2H), 3.89 (s, 3H), 2.91 (m, 2H), 2.70 (m, 2H), 2.70 (d, J=4.48 Hz, 1H); ¹³C NMR (CDCl₃): 163.8, 137.5, 130.4, 129.6, 129.1, 128.8, 126.9, 114.5, 56.0, 41.6, 37.9, 33.1.

5. (S)-2-Benzyl-1-(4-fluorobenzenesulfonyl)aziridine

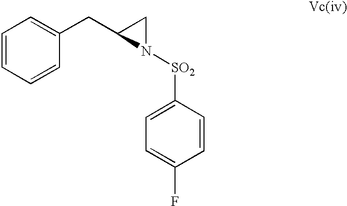

Slightly yellow colored solid; ¹H NMR (CDCl₃): 7.79 (m, 2H), 7.11 (m, 7H), 2.98 (m, 2H), 2.78 (d, J=6.8 Hz, 1H), 2.62(m, 1H), 2.22(d, J=4.51 Hz, 1H); ¹³C NMR (CDCl₃): 167.6, 164.2, 137.3, 131.0, 130.9, 129.0, 128.9, 127.0, 116.7, 116.4,42.2,37.9,33.3.

6. (S)-2-Benzyl-1-(propane-2-sulfonyl)aziridine

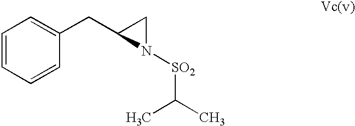

Viscous oil; ¹H NMR (CDCl₃): 7.30(m, 5H), 2.95(m, 4H), 2.64(d, J=6.69 Hz, 1H), 2.15(d, J=4.37 Hz, 1H), 1.38, 1.35 (2d, J=2.75 Hz, 2.81 Hz, 6H); ¹³C NMR (CDCI₃): 137.2, 129.3, 129.0, 127.3, 53.7, 40.3, 37.9, 32.8, 16.8, 16.4.

7. (S)-2-Isopropyl-1-(toluene-4-sulfonyl)aziridine

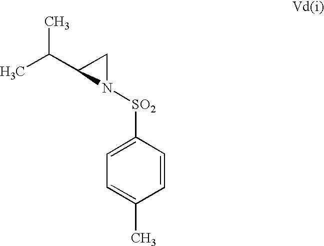

White solid; ¹H NMR (CDCl₃): 7.84 (d, J=8.28 Hz, 2H), 7.35(d, J=7.97 Hz, 2H), 2.63(d, J=7.0 Hz, 1H), 2.53(m, 1H), 2.46(s, 3H), 2.11(d, J=4.6 Hz, 1H), 1.41(m, 1H), 0.93(d, J=6.83 Hz, 3H), 0.75(d, J=6.69 Hz, 3H) ¹³C NMR (CDCl₃): 144.8, 135.5, 129.9, 128.4, 46.6, 33.1, 30.5, 22.0, 19.9, 19.4.

8. (S)-1-Benzenesulfonyl-2-isopropylaziridine

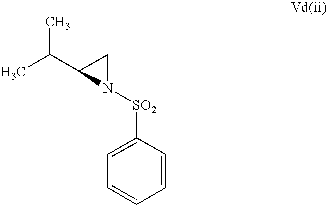

Viscous oil; ¹H NMR (CDCl₃): 7.95 (m, 2H), 7.63(m, 3H), 2.63(d, J=7.0 Hz, 1H), 2.53 (m, 1H), 2.12 (d, J=4.6 Hz, 1H), 1.42(m, 1H), 0.89(d, J=6.84 Hz, 3H), 0.77(d, J=6.7 Hz, 3H); ¹³C NMR (CDCl₃): 138.4, 133.9, 129.4, 128.4, 46.7, 33.2, 30.5, 19.8, 19.4.

9. (S)-1-(4-Methoxybenzenesulfonyl)-2-isopropylaziridine

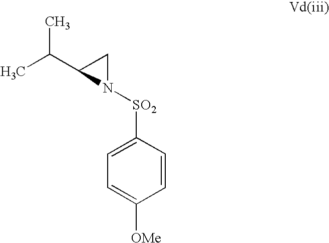

Viscous oil; ¹H NMR (CDCl₃): 7.84 (d, J=8.86 Hz, 2H), 7.01(d, J=8.86 Hz, 2H), 3.86(s, 3H), 2.58(d, J=6.99 Hz, 1H), 2.47 (m, 1H), 2.08(d, J=4.56 Hz, 1H), 1.37 (m, 1H), 0.88(d, J=6.82 Hz, 3H), 0.77(d, J=6.68 Hz, 3H); ¹³C NMR (CDCl₃): 163.9, 130.5, 129.7, 114.5, 56.0, 46.5, 32.9, 30.4, 19.8, 19.4.

10. (S)-1-(4-Fluorobenzenesulfonyl)-2-isopropylaziridine

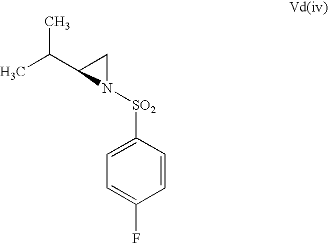

Viscous oil; ¹H NMR (CDCl₃): 7.97 (m, 2H), 7.22 (m, 2H), 2.63(d, J=7.03 Hz, 1H), 2.52(m, 1H), 2.12(d, J=4.64 Hz, 1H), 1.40 (m, 1H), 0.90(d, J=6.84 Hz, 3H), 0.78 (d, J=6.69 Hz, 3H); ¹³C NMR (CDCl₃): 167.7, 164.3, 131.3, 131.1, 116.8, 116.5, 46.9, 33.3, 30.4,19.8,19.4.

11. (S)-2-Isopropyl-1-(propane-2-sulfonyl)aziridine

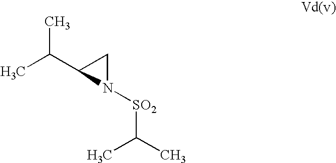

Viscous oil; ¹H NMR (CDCl₃): 3.26 (m, 1H), 2.52 (m, 1H), 2.10 (m, 1H), 1.53 (m, 1H), 1.44 (d, J=5.06 Hz, 3H), 1.42 (d, J=5.11 Hz, 3H), 1.01 (d, J=6.72 Hz, 3H), 0.95 (d, J=6.84 Hz, 3H); ¹³C NMR (CDCl₃): 53.5, 44.2, 32.9, 30.2, 19.9, 19.1, 16.8, 14.4.

12. (S,S)-2-sec-Butyl-1-(toluene-4-sulfonyl)aziridine

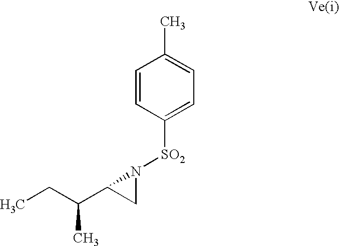

Viscous oil; ¹H NMR (CDCl₃): 7.80 (d, J=8.3 Hz, 2H), 7.31 (d, J=8 Hz, 2H), 2.56 (m, 2H), 2.42(s, 3H), 2.05 (m, 1H), 1.36 (m, 1H), 1.13(m, 2H), 0.88 (d, J=6.65 Hz, 3H), 0.78 (t, J=7.30 Hz, 3H); ¹³C NMR (CDCl₃): 144.8, 135.3, 129.9, 128.4, 45.5, 36.9, 33.0, 27.4, 21.9, 15.9, 11.1.

13. (S,S)-1-Benzenesulfonyl-2-sec-butylaziridine

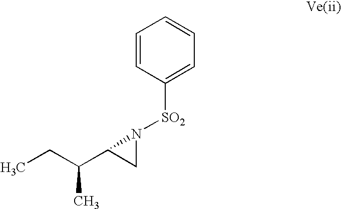

Viscous oil; ¹H NMR (CDCl₃): 7.94 (m, 2H), 7.56 (m, 3H), 2.60 (m, 2H), 2.07 (m, 1H), 1.32 (m, 1H), 1.15(m, 2H), 1.05 (d, J=6.63 Hz, 3H), 0.78 (t, J=7.37 Hz, 3H); ¹³C NMR (CDCl₃): 138.3, 133.9, 129.3, 128.3, 45.6, 36.9, 33.1, 27.4, 15.9, 11.1.

14. (S,S)-2-sec-Butyl-1-(4-methoxybenzenesulfonyl)aziridine

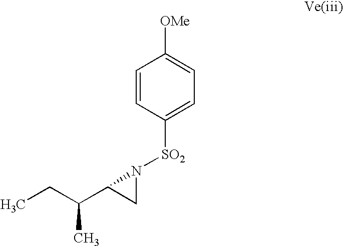

Viscous oil; ¹H NMR (CDCl₃): 7.81 (d, J=8.97 Hz, 2H), 6.96 (d, J=8.88 Hz, 2H), 3.83 (s, 3H), 2.51 (m, 2H), 2.01 (m, 1H), 1.35 (m, 1H), 1.12(m, 2H), 0.84 (d, J=6.6 Hz, 3H), 0.77(t, J=7.33 Hz, 3H); ¹³C NMR (CDCl₃): 163.9, 130.5, 129.8, 114.5, 56.0, 45.5, 36.9, 32.9, 27.4, 15.9, 11.1.

15. (S,S)-2-sec-Butyl-1-(4-fluorobenzenesulfonyl)aziridine

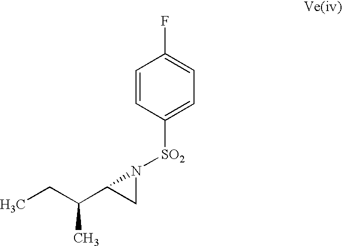

Viscous oil; ¹H NMR (CDCl₃): 8.01 (m, 2H), 7.21 (m, 2H), 2.56 (m, 2H), 2.01 (m, 1H), 1.41(m, 1H), 1.15(m, 2H), 0.86 (d, J=6.7 Hz, 3H), 0.78(t, J=7.45 Hz, 3H); ¹³C NMR (CDCl₃): 167.7, 164.3, 134.7, 134.6, 131.2, 131.1, 116.8, 116.5, 45.8, 36.9, 33.2, 27.4, 15.9, 11.1.

16. (S,S)-2-sec-Butyl-1-(propane-2-sulfonyl)aziridine

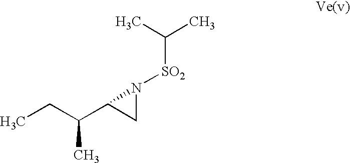

Viscous oil; ¹H NMR (CDCl₃): 3.25 (m, 1H), 2.54 (m, 2H), 2.07 (m, 1H), 1.61 (m, 1H), 1.42 (d, J=2.79 Hz, 3H), 1.41 (d, J=2.9 Hz, 3H), 1.29 (m, 2H), 0.91 (m, 6H); ¹³C NMR (CDCl₃): 53.7, 43.1, 36.8, 33.0, 27.6, 16.9, 16.5, 15.6, 11.3.

17. (S)-2-Cyclohexylmethyl-1-(toluene-4-sulfonyl)aziridine

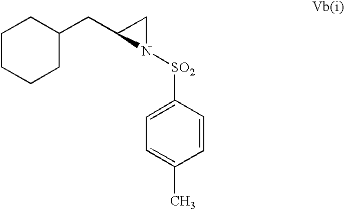

Viscous oil; ¹H NMR (CDCl₃): 7.83 (d, J=8.28 Hz, 2H), 7.34 (d, J=8.07 Hz, 2H), 2.82(m, 1H), 2.66(d, J=6.99 Hz, 1H), 2.45 (s, 3H), 2.03(d, J=4.57 Hz, 1H), 1.62(m, 5H), 1.27(m, 8H); ¹³C NMR (CDCl₃): 144.7, 135.5, 129.9, 128.2, 39.4, 39.2, 36.3, 34.0, 33.6, 32.8, 26.6, 26.4, 26.3.

18. (S)-2-Cyclohexylmethyl-1-(benzenesulfonyl)aziridine

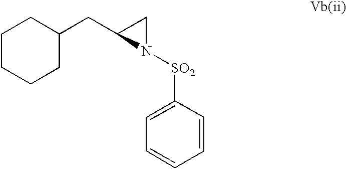

Viscous oil; ¹H NMR (CDCl₃): 7.95 (m, 2H), 7.62 (m, 1H), 7.55 (m, 2H), 2.65 (d, J=7 Hz), 2.02 (d, J=4.6 Hz), 1.75 (m, 1H), 1.65 (m, 5H), 1.22 (m, 6H), 0.82 (m, 2H); ¹³C NMR (CDCl₃): 138.5, 133.9, 129.4, 128.3, 39.5, 39.4, 36.4, 34.3, 33.7, 32.9, 26.6, 26.4, 26.3.

19. (S)-2-Cyclohexylmethyl-1-(4-methoxybenzenesulfonyl)aziridine

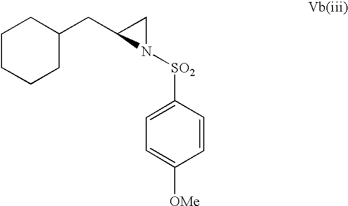

Viscous oil; ¹H NMR (CDCl₃): 7.85 (d, J=8.76 Hz, 2H), 7.01(d, J=8.9 Hz, 2H), 3.86 (s, 3H), 2.75 (m, IH), 2.62 (d, J=7 Hz, 1H), 2.01(d, J=4.5 Hz, IH), 1.62 (m, 5H), 1.22 (m, 6H), 0.95 (m, 2H); ¹³C NMR (CDCl₃): 163.9, 130.4, 129.9, 114.6, 56.0, 39.4, 39.2, 36.4, 34.1, 33.7, 32.9, 26.6, 26.4, 26.3.

20. (S)-2-Cyclohexylmethyl-1-(4-fluorobenzenesulfonyl)aziridine

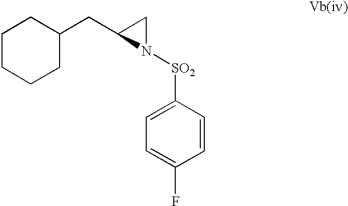

Viscous oil; ¹H NMR (CDCl₃): 7.95(m, 2H), 7.23 (m, 2H), 2.81(m, 1H), 2.66 (d, J=7 Hz, 1H), 2.02 (d, J=4.6 Hz, 1H), 1.61(m, 5H), 1.22 (m, 6H), 0.92 (m, 2H); ¹³C NMR (CDCl₃): 167.7, 164.3, 134.8, 134.7, 131.2, 131.0, 116.8, 116.5, 39.6, 39.4, 36.5, 34.4, 33.7, 32.9, 26.6, 26.4, 26.3.

21. (S)-2-Cyclohexylmethyl-1-(propane-2-sulfonyl)aziridine

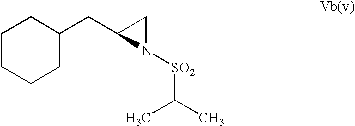

Colorless oil; ¹H NMR (CDCl₃): 3.27(m, 1H), 2.75(m, 1H), 2.60 (d, J=6.92 Hz, 1H), 2.04 (d, J=4.61 Hz, 1H), 1.72 (m, 6H), 1.46 (d, J=6.88 Hz, 6H), 1.25 (m, 7H); ¹³C NMR (CDCl₃): 53.9, 39.6, 37.7, 36.4, 34.4, 33.8, 33.2, 26.7, 26.5, 26.4, 16.8, 16.6.

22. (S)-1-Benzenesulfonyl-2-isobutylaziridinc

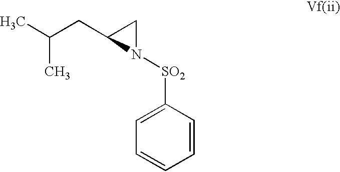

Colorless oil; ¹H NMR (CDCl₃): 7.99-7.46 (m, 5H), 2.86-2.74 (m, 1H), 2.69-2.61 (d, J=7 Hz, 1H), 2.08-2.00 (d, J=4.6 Hz, 1H), 1.68-1.50 (m, 1H), 1.33 (t, J=6.8 Hz, 2H), 0.90 (d, J=2 Hz, 3H), 0.86 (d, J=2 Hz, 3H); ¹³C NMR (CDCl₃): 138.6, 133.9, 129.4, 128.3, 40.7, 39.5, 34.5, 27.1, 23.1, 22.3.

23. (S)-2-Isobutyl-1-(toluene-4-sulfonyl)aziridine

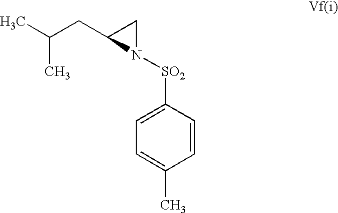

Colorless oil; ¹H NMR (CDCl₃): 7.86-7.77(d, J=8.3 Hz, 2H), 7.38-7.28(d, J=8.2 Hz, 2H), 2.84-2.71 (m, 1H), 2.69-2.58 (d, J=7 Hz, 1H), 2.43 (s, 1H), 2.05-1.99 (d, J=4.6 Hz, 1H), 1.69-1.53 (m, 1H), 1.38-1.28 (t, J=6.8 Hz, 2H), 0.90 (d, J=2 Hz, 3H), 0.86 (d, J=2 Hz, 3H); ¹³C NMR (CDCl₃): 144.8, 135.6, 130.0, 128.3, 40.8, 39.4, 34.4, 27.1, 23.1, 22.3, 22.0.

24. (S)-1-(4-Fluorobenzenesulfonyl)-2-isobutylaziridine

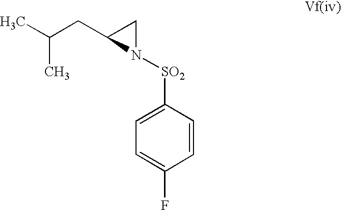

Colorless oil; ¹H NMR (CDCl₃): 8.02-7.93 (m, 2H), 7.28-7.17(m, 2H), 2.89-2.76(m, 1H), 2.69-2.62(d, J=7 Hz, 1H), 2.09-2.02(d, J=4.6 Hz, 1H),1.69-1.50(m, 1H), 1.39-1.28(t, J=6.8 Hz, 2H), 0.90 (d, J=1.8 Hz, 3H), 0.86 (d, J=1.8 Hz, 3H); ¹³C NMR (CDCl₃): 168.9, 165.5, 136.0, 135.9, 132.4, 132.3, 118.1, 117.8, 41.9, 41.0, 35.8, 28.4, 24.3, 23.5.5.

25. (S)-1-(4-Methoxybenzenesulfonyl)-2-isobutylaziridine

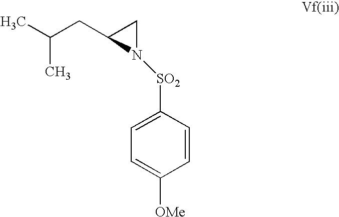

Colorless oil; ¹H NMR (CDCl₃): 7.93-7.77 (dd, J=2 Hz, 2H), 7.09-6.88 (dd, J=2 Hz, 2H), 3.86 (s, 3H), 2.80-2.67(m, 1H), 2.62-2.55(d, J=7 Hz, 1H), 2.04-1.97(d, J=5 Hz, 1H), 1.69-1.51(m, 1H), 1.37-1.26(t, J=6.8 Hz, 2H), 0.90 (d, J=2 Hz, 3H), 0.86 (d, J=2 Hz, 3H); ¹³C NMR (CDCl₃):164, 130.4, 129.9, 114.6, 56.0, 53.9, 40.7, 39.3, 34.2, 27.1, 23.1, 22.3.

26. (S)-2-Isobutyl-1-(propane-2-sulfonyl)aziridine

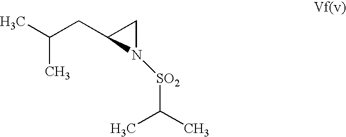

Colorless oil; ¹H NMR (CDCl₃): 3.36-3.20(m, 1H), 2.76-2.65(m, 1H), 2.62-2.55 (d, J=7 Hz, 1H), 2.09-2.02(d, J=4.6 Hz, 1H), 1.88-1.71(m, 1H), 1.60-1.49 (m, 1H), 1.48-1.41(dd, J=0.6 Hz, J=0.7 Hz, 6H), 1.40-1.23(m, 1H), 0.90 (d, J-2 Hz, 3H), 0.86 (d, J=2 Hz, 3H); ¹³C NMR (CDCl₃): 53.8, 40.9, 37.6, 34.4, 27.0, 23.1, 22.5, 16.7, 16.6.

The invention and manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A method for preparing a chiral aziridine comprising:
(a) treating an α-amino alcohol with a sulfuric acid to form a sulfate ester; and
(b) cyclizing the sulfate ester in the presence of base to form the chiral aziridine.

2. The method according to claim 1 wherein the chiral aziridine is 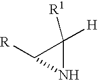
wherein [symbol]
is a bond representing either (S) or (R) configuration with respect to the carbon atom to which it is attached;
R is hydrogen, or
lower alkyl optionally substituted with one, two, three or four groups independently selected from halogen, aryl, cycloalkyl, heteroaryl, lower alkoxy, —C(O)-alkyl, —C(O)NH-alkyl, C(O)N-dialkyl, nitro, alkenyl or alkynyl, or
aryl or heteroaryl optionally substituted with one, two, three or four groups independently selected from halogen, lower alkyl, lower alkoxy, —C(O)— alkyl, —C(O)NH-alkyl, C(O)N-dialkyl, nitro, alkenyl or alkynyl; and
R¹ is hydrogen or lower alkyl.

3. The method of claim 1 wherein the α-amino alcohol is treated with the sulfuric acid at a temperature of from between 0° C. and 5° C.

4. The method according to claim 3 wherein the temperature is about 5° C.

5. The method according to claim 3 wherein the α-amino alcohol is treated with the sulfuric acid at a pressure of from between 0.5 mm/Hg to 5 mm/Hg.

6. The method according to claim 5 wherein the pressure is from between 1 mm/Hg to 3 mm/Hg.

7. The method according to claim 6 wherein the pressure is around 2 mm/Hg.

8. The method according to claim 5 wherein the α-amino alcohol is treated with the sulfuric acid in a suitable solvent.

9. The method according to claim 8 wherein the solvent is selected from water and diethyl ether.

10. The method according to claim 1 wherein the sulfate ester is cyclized in a second solvent.

11. The method according to claim 10 wherein the second solvent is a protic solvent.

12. The method according to claim 11 wherein the second solvent is water.

13. The method according to claim 11 wherein the base is selected from an alkali metal base and an alkaline earth metal base.

14. The method according to claim 13 wherein the base is alkaline metal base.

15. The method according to claim 14 wherein the base is selected from sodium hydroxide or potassium hydroxide.

16. The method according to claim 1 further comprising protecting the chiral aziridine to afford a chiral N-protected aziridine.

17. The method according to claim 16 wherein the chiral aziridine is treated with a sulfonyl chloride to afford a chiral N-sulfonylaziridine.

18. A method of preparing a chiral aziridine of the formula 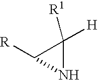
wherein [symbol]
is a bond representing either (S) or (R) configuration with respect to the carbon atom to which it is attached;
R is hydrogen, or
lower alkyl optionally substituted with one, two, three or four groups independently selected from halogen, aryl, cycloalkyl, heteroaryl, lower alkoxy, —C(O)-alkyl, —C(O)NH-alkyl, C(O)N-dialkyl, nitro, alkenyl or alkynyl, or
aryl or heteroaryl optionally substituted with one, two, three or four groups independently selected from halogen, lower alkyl, lower alkoxy, —C(O)— alkyl, —C(O)NH-alkyl, C(O)N-dialkyl, nitro, alkenyl or alkynyl; and
R¹ is hydrogen or lower alkyl,
the method comprising
(a) treating an α-amino alcohol of the formula 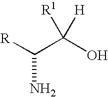
with a sulfuric acid to form a sulfate of the formula 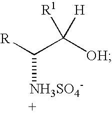
(b) heating the sulfate under reduced pressure to form a sulfate ester of the formula 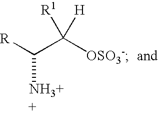
(c) cyclizing the sulfate ester in the presence of base to form the chiral aziridine.

19. The method according to claim 18 wherein the α-amino alcohol is

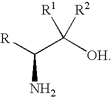

20. The method according to claim 18 wherein the α-amino alcohol is

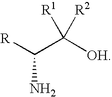

21. The method according to claim 18 further comprising treating the chiral aziridine with a sulfonyl chloride of the formula
R³SO₂Cl
wherein
R³ is lower alkyl optionally substituted with one, two or three groups independently selected from halogen, aryl, cycloalkyl, heteroaryl, lower alkoxy, —C(O)-alkyl, —C(O)NH-alkyl, C(O)N-dialkyl, —C(O)O-alkyl, cyano, nitro, alkenyl or alkynyl, or
R³ is aryl optionally substituted with one, two, three or four groups independently selected from halogen, lower alkyl, lower alkoxy, —C(O)-alkyl, amino, mono- or dialkylamino, mercapto, alkylthiol, —C(O)NH-alkyl, C(O)N-dialkyl, nitro, alkenyl or alkynyl,
to afford a chiral N-sulfonylaziridine of the formula [chemical structure]
